(19) United States
(12) Patent Application Publication
(10) Pub. No.: US 2007/0191872 A1
(43) Pub. Date: Aug. 16, 2007

(54) MEMBRANE EYELET

(75) Inventor: Mark Stiger, Windsor, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/739,404

(22) Filed: Apr. 24, 2007

Related U.S. Application Data
(63) Continuation-in-part of application No. 10/423,147, filed on Apr. 24, 2003.

(51) Int. Cl. A61B 17/08

(52) U.S. Cl. 606/153

(57) ABSTRACT

A structure and method for deploying an eyelet in a membrane, where the eyelet includes: a waist section; a first anchor section coupled to and flared from the waist section; and a second anchor section coupled to and flared from the waist section. The eyelet is deployed such that the waist section is located within a membrane opening of the membrane thus keeping the membrane opening open. Further, the membrane is sandwiched between the first and second anchor sections thus anchoring the eyelet to the membrane.

### TECHNICAL FIELD

The present invention relates to a medical device and method. More particularly, the present invention relates to a device and method for maintaining an opening or orifice in a septum (or tissue membrane).

### BACKGROUND OF THE INVENTION

Non-communicating hydrocephalus is a condition that results in the enlargement of the ventricles caused by abnormal accumulation of cerebrospinal fluid (CSF) within the cerebral ventricular system.

In non-communicating hydrocephalus there is an obstruction at some point in the ventricular system. The cause of non-communicating hydrocephalus usually is a congenital abnormality, such as stenosis of the aqueduct of Sylvius, congenital atresia of the foramina of the fourth ventricle, or spina bifida cystica. There are also acquired versions of hydrocephalus that are caused by a number of factors including subarachnoid or intraventricular hemorrhages, infections, inflammation, tumors, and cysts.

The main treatment for hydrocephalus is venticuloperitoneal (VP) shunts. The VP shunts are catheters that are surgically lowered through the skull and brain. The VP shunts are then positioned in the lateral ventricle. The distal end of the catheter is tunneled under the skin and positioned in the peritoneal cavity of the abdomen, where the CSF is absorbed.

However, the VP shunts have an extremely high failure rate, e.g., in the range of 30 to 40 percent. Failure includes clogging of the catheter, infection, and faulty pressure valves or one-way valves.

Another treatment for non-communicating hydrocephalus is the procedure known as an endoscopic third ventriculostomy (ETV). This procedure involves forming a burr hole in the skull. A probe is passed through the burr hole, through the cerebral cortex, through the underlying white matter and into the lateral and third ventricles. The probe is then used to poke (fenestrate) a hole in the floor of the third ventricle and underlying membrane of Lillequist.

To verify that the procedure is successful, i.e., that a hole is formed in the floor of the third ventricle and the underlying membrane of Lillequist, the patient is observed with a magnetic resonance imaging (MRI) device after the probe poke. The MRI device is used to verify a flow of CSF through the hole in the floor of the third ventricle.

If the MRI device is unable to detect the flow of CSF, a determination is made that a hole in the floor of the third ventricle was not formed, and the ETV procedure is repeated.

Since the MRI device is typically located at a separate location, the ETV procedure typically requires the patient to be moved from location to location. This, in turn, increases the procedure time as well as the expense and complexity of the ETV procedure.

Further, even after successfully forming a hole in the floor of the third ventricle, the hole sometimes closes, typically within two weeks to two months after the ETV procedure. In this event, the patient will have to undergo another ETV procedure or risk serious injury or death.

### SUMMARY OF THE INVENTION

The current invention discloses a membrane eyelet deployed in a tissue membrane. The membrane eyelet includes a waist section; a first anchor section coupled to and flared from the waist section; and a second anchor section coupled to and flared from the waist section.

The membrane eyelet is deployed such that the waist section is located within a hole that is formed in the tissue membrane. Membrane engaging struts or annular rings help to keep the hole from closing. Further, the tissue membrane is sandwiched between the first and second anchor sections. Thus, the membrane eyelet resides generally coplanar with the tissue membrane. The waist section keeps the opening, through which fluid or air can pass, open.

### BRIEF DESCRIPTION OF THE DRAWINGS

FIG. 1 is a side view showing the front half of a membrane eyelet, prior to deployment, in one embodiment according to the present invention;

FIG. 2 is a front view of a membrane eyelet deployed in a tissue membrane viewed in the direction II of FIG. 3A, after the membrane eyelet of FIG. 1 has been deployed in a tissue membrane;

FIG. 2B is a partial cross-sectional view taken at III-III of FIG. 2 of the membrane eyelet deployed within the tissue membrane;

FIG. 3 is a partial cross-sectional view of another membrane eyelet deployed within a tissue membrane;

FIG. 4 is a side view of a membrane eyelet, prior to deployment, in one embodiment according to the present invention;

FIG. 5 is a front view of the membrane eyelet viewed in the direction V of FIG. 6, after the membrane eyelet of FIG. 4 has been deployed within a tissue membrane;

FIG. 6 is a cross-sectional view taken at VI-VI of FIG. 5 of the membrane eyelet deployed within the tissue membrane;

FIG. 7 is a side view of a membrane eyelet, prior to deployment, in one embodiment according to the present invention;

FIG. 8 is a partial cross-sectional view of the membrane eyelet of FIG. 7, after deployment within a tissue membrane;

FIG. 9 is a side view of a membrane eyelet, prior to deployment, in one embodiment according to the present invention;

FIG. 10 is a front view of the membrane eyelet of FIG. 9 deployed in a tissue membrane;

FIG. 11 is a side view of a membrane eyelet, prior to deployment, in one embodiment according to the present invention;

FIG. 12 is a front view of the membrane eyelet of FIG. 11, after deployment within a tissue membrane;

FIG. 13A is a cross-sectional view of a bridge of the membrane eyelet of FIG. 1 taken at XIII-XIII;

FIGS. 13B and 13C are cross-sectional views of bridges of membrane eyelets similar to the membrane eyelet of FIG. 1;

FIG. 14 is a cross-sectional view of the membrane eyelet of FIG. 1 taken at XIV-XIV;

FIG. 15 is a cross-section view of a human cranium during an endoscopic third ventriculostomy (ETV) procedure using an endoscopic third ventriculostomy probe in one embodiment according to the present invention;

FIGS. 16 through 19 are illustrations of another embodiment of a membrane eyelet according to the current invention.

### DETAILED DESCRIPTION

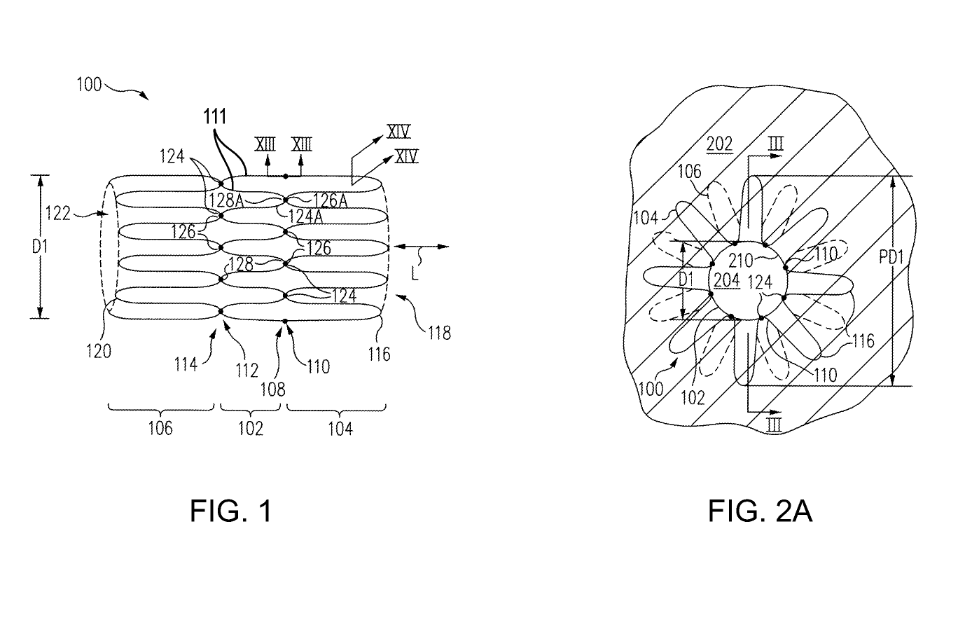

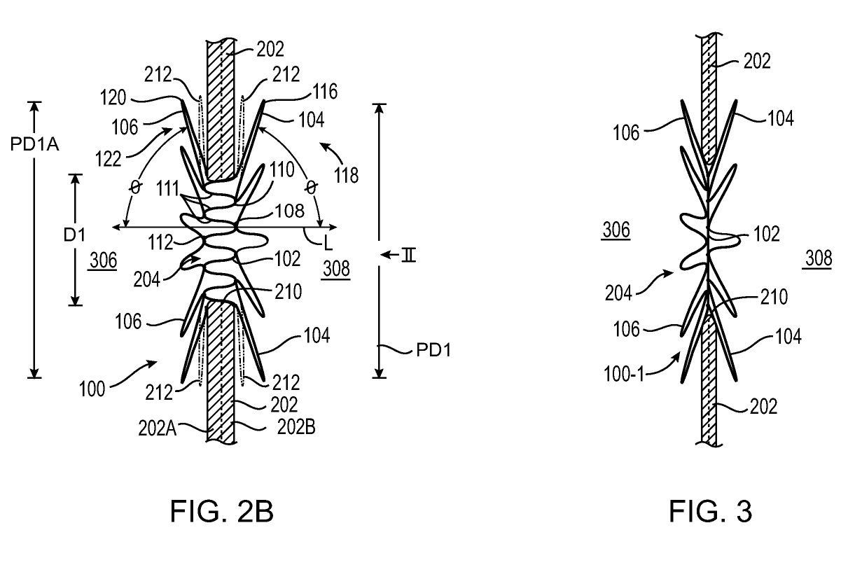

The invention will now be described by reference to the figures wherein like numbers refer to like structures. FIG. 1 is a side view of a membrane eyelet 100, prior to deployment. In accordance with one embodiment of the present invention, a membrane eyelet 100 (FIGS. 2A and 2B) deployed in a tissue membrane 202 includes: a waist section 102; a first anchor section 104 coupled to and flared from waist section 102; and a second anchor section 106 coupled to and flared from waist section 102.

Membrane eyelet 100 is deployed such that waist section 102 is located within an opening 204 in tissue membrane 202. Further, tissue membrane 202 is sandwiched between first and second anchor sections 104, 106. Waist section 102 keeps membrane opening 204 through which fluid or air can pass open. By sandwiching tissue membrane 202, the first and second anchor sections 104,106 anchor membrane eyelet 100 to tissue membrane 202.

In FIG. 1, only the near side cylindrical surface of membrane eyelet 100 is illustrated for clarity of illustration, however, it is to be understood that parts of the far side cylindrical surface of membrane eyelet 100 would also be visible. The membrane eyelet 100 includes a waist section 102, a right, e.g., first, anchor section 104, and a left, e.g., second, anchor section 106. The anchor sections 104, 106 and the waist section 102 are formed from generally serpentine rings. Waist section 102 is between and directly coupled to first anchor section 104 and second anchor section 106.

More particularly, waist section 102 includes a right, e.g., first, edge 108 coupled to a left, e.g., waist section edge 110 of first anchor section 104. Further, waist section 102 includes a left, e.g., second, edge 112 coupled to a right, e.g., waist section edge 114 of second anchor section 106. The first and second edges of the waist section are defined by the ends of the serpentine ring, and a plurality of struts 111 extend from the first edge to the second edge of the waist section.

First anchor section 104 further includes a right, e.g., outer edge 116 as represented by the dashed line forming a proximal, e.g., first, end 118 of membrane eyelet 100. Second anchor section 106 further includes a left, e.g., outer edge 120 as represented by the dashed line forming a distal, e.g., second, end 122 of membrane eyelet 100.

Prior to deployment, as shown in FIG. 1, membrane eyelet 100 is cylindrical in shape having a longitudinal axis L. More particularly, waist section 102, first anchor section 104 and second anchor section 106 are rings, sometimes called ring shaped structures. In accordance with one embodiment, membrane eyelet 100 has a first radial diameter D1 prior to deployment.

FIG. 2A is a front view of membrane eyelet 100 viewed from the direction II of FIG. 2B, after deployment within a tissue membrane 202. FIG. 2B is a partial cross-sectional view taken along III-III of FIG. 2A of membrane eyelet 100 deployed within tissue membrane 202. As shown in FIGS. 2A and 2B, membrane eyelet 100 is deployed to maintain the patency of an opening 204, sometimes called an aperture, pathway, or orifice, that has been created in a tissue membrane 202. Opening 204 forms a pathway through which fluid or air can pass from first region 306 to second region 308 or vice versa.

In one embodiment, the membrane is the floor of the third ventricle and the membrane eyelet is used to treat hydrocephalus. In accordance with this embodiment, cerebrospinal fluid (CSF) from the 3rd ventricle flows through an opening and the membrane eyelet into the interpeduncular cistern, thus relieving pressure from the 3rd ventricle.

As another example, a membrane eyelet can be used to support an opening through which air flows from a prosthetic airway through to the main brachial airway.

In one embodiment, the membrane is a single integral membrane. However, in another embodiment, tissue membrane 202 is formed of two or more membranes (illustratively labeled 202A and 202B and separated by the dashed line in FIG. 2B).

The membranes can be membranes that normally abut each other, or they can be separate such that there is generally a space between the membranes and they are held together by membrane eyelet. For example, opposing openings can be formed in two adjacent blood vessels, arteries, veins or adjacent membranes in the body. In accordance with the invention, a membrane eyelet can be used to provide fluid transfer such as pressure relief to/from a vessel.

Referring now to FIGS. 1 and 2B one embodiment of a membrane eyelet includes a waist section 102 that is formed from a generally serpentine ring. The struts 111 in the waist section 102 directly contact the interior edges 210 of the opening that has been created in the tissue membrane 202 and keep the opening from closing. Opening interior edge 210 defines opening 204. Waist section 102 prevents opening interior edge 210 from contracting and thus prevents opening 204 from closing. Stated another way, waist section 102 keeps opening 204 open thus preventing constriction of the pathway through which fluid or air can pass from first region 306 to second region 308 or vice versa.

Anchor sections 104 and 106 are flared upon deployment of membrane eyelet 100 to engage the tissue membrane 202 thus anchoring membrane eyelet 100 to tissue membrane 202. In the embodiment depicted in FIG. 2B, the waist section 102 remains cylindrical. However, first anchor section 104 and second anchor section 106 are flared outwards, sometimes called winged, from waist section 102 to sandwich tissue membrane 202 between first anchor section 104 and second anchor section 106. Stated another way, first anchor section 104 and second anchor section 106 wrap around tissue membrane 202 during deployment of membrane eyelet 100. Accordingly, after deployment, membrane eyelet 100 is said to have an membrane eyelet shape.

Prior to deployment, the membrane eyelet is in a delivery configuration wherein it is crimped to the surface of an expandable balloon or another delivery device. The membrane eyelet is then delivered to an opening in a tissue membrane and deployed. In the embodiment depicted in FIG. 1, after deployment of membrane eyelet 100, waist section 102 has a radial deployed diameter D1. First anchor section 104 has radial diameter D1 at left edge 110 and a peripheral radial diameter PD1 at right edge 116. Peripheral radial diameter PD1 at right edge 116 of first anchor section 104 is greater than radial diameter D1 at left edge 110 of first anchor section 104 such that first anchor section 104 flares outwards, sometimes called increases in radial diameter, from left edge 110 to right edge 116.

To further illustrate, second anchor section 106 has radial diameter D1 at right edge 114 and a peripheral radial diameter PD1A at left edge 120. Since peripheral radial diameter PD1A at left edge 120 of second anchor section 106 is greater than radial diameter D1 at right edge 114 of second anchor section 106, second anchor section 106 flares outwards, sometimes called increases in radial diameter, from right edge 114 to left edge 120.

By sandwiching the tissue membrane 202 between first anchor section 104 and second anchor section 106, unintentional detachment of membrane eyelet 100 from tissue membrane 202 is avoided. Generally, an angle θ between longitudinal axis L and planes or conical surfaces defined by anchor sections 104 and 106 is sufficiently large to create overlap or enlargement to prevent unintentional the detachment of membrane eyelet 100 from tissue membrane 202.

As shown in FIG. 2B, angle θ is less than 90° in one embodiment such that anchor sections 104 and 106 define conical surfaces. Specifically, first anchor section 104 and/or second anchor section 106 are spaced apart from tissue membrane 202 and do not directly contact tissue membrane 202 or only contact tissue membrane 202 directly adjacent waist section 102. However, first anchor section 104 and/or second anchor section 106 form stops that limit the amount of longitudinal movement (left and/or right movement in the view of FIG. 2B) of membrane eyelet 100.

To illustrate, membrane eyelet 100 is allowed some degree of longitudinal movement in the left direction until first anchor section 104 is pressed into tissue membrane 202 thus preventing further longitudinal movement. Similarly, membrane eyelet 100 is allowed some degree of longitudinal movement in the right direction until second anchor section 106 is pressed into tissue membrane 202 thus preventing further longitudinal movement. However, in yet another embodiment, first anchor section 104 and second anchor section 106 are pressed into tissue membrane 202 upon deployment of membrane eyelet 100 thus preventing any longitudinal motion of membrane eyelet 100.

Further, as indicated by the dashed lines 212, angle θ is equal to or greater than 90° in one embodiment. When angle θ is equal to 90°, first anchor section 104 and second anchor section 106 define planes perpendicular to longitudinal axis L. In accordance with this embodiment, first anchor section 104 and second anchor section 106 are pressed into direct contact with tissue membrane 202.

To deploy membrane eyelet 100, membrane eyelet 100 is inserted into opening 204 such that waist section 102 is located within opening 204. Membrane eyelet 100 is radially expanded to sandwich tissue membrane 202 between first anchor section 104 and second anchor section 106 thus securing waist section 102 within opening 204. In one embodiment, membrane eyelet 100 is radially expanded using a dilation balloon or by a longitudinal compression of a mesh of juxtaposed fibers.

In another embodiment, membrane eyelet 100 is self-expanding where membrane eyelet 100 is constrained within a sheath. Retraction of the sheath exposes membrane eyelet 100, which self-expands. Use of a sheath to deploy a self-expanding device is well known to those of skill in the art and so is not discussed further.

In one embodiment, first anchor section 104 and second anchor section 106 are selectively expandable relative to waist section 102, i.e., can be radially expanded more than waist section 102. Illustratively, waist section 102 has greater strength than first anchor section 104 and second anchor section 106 such that application of an outwards force, e.g., from a dilation balloon, selectively expands and flares first anchor section 104 and second anchor section 106 relative to waist section 102. To further illustrate, in the example when membrane eyelet 100 is self-expanding, first anchor section 104 and second anchor section 106 are configured to expand more than waist section 102.

Referring again to FIG. 1, first anchor section 104 is a serpentine ring, sometimes called crown. First anchor section 104 has a pattern, and this pattern is sometimes called a serpentine pattern, an alternating repeating pattern, or a zigzag pattern.

In the depicted embodiment, the serpentine pattern extends around a cylindrical surface having longitudinal axis L. Second anchor section 106 is essentially identical to first anchor section 104 though rotationally offset. The rotational offset can seen in FIG. 2A wherein the serpentine structure of the second anchor section 106 is shown as dotted lines.

Further, waist section 102 has a pattern, and this pattern is sometimes called a serpentine pattern, an alternating repeating pattern, or a zigzag pattern. More particularly, the serpentine pattern extends around a cylindrical surface having longitudinal axis L. Waist section 102 has the same pattern as anchor sections 104, 106, but the height, sometimes called amplitude, of the serpentine pattern of waist section 102 is less than the height of the serpentine patterns of anchor sections 104, 106. In another embodiment, the height of the serpentine pattern of waist section 102 is equal to or greater than the height of the serpentine patterns of anchor sections 104, 106.

Anchor sections 104, 106 are connected to waist section 102 by bridges 124. Bridges 124 extend between peaks 126 of the serpentine patterns of anchor sections 104, 106 and peaks 128 of the serpentine pattern of waist section 102. Peaks 126 and 128 are sometimes called minima/maxima of the serpentine patterns of anchor sections 104,106 and waist section 102, respectively. Bridges 124 can be formed at each adjacent peak 126 and 128, or only at some (fewer than all) of peaks 126 and 128.

To illustrate, a first bridge 124A of the plurality of bridges 124 extends between a first peak 126A of the plurality of peaks 126 of the serpentine pattern of first anchor section 104 and a first peak 128A of the plurality of peaks 128 of the serpentine pattern of waist section 102.

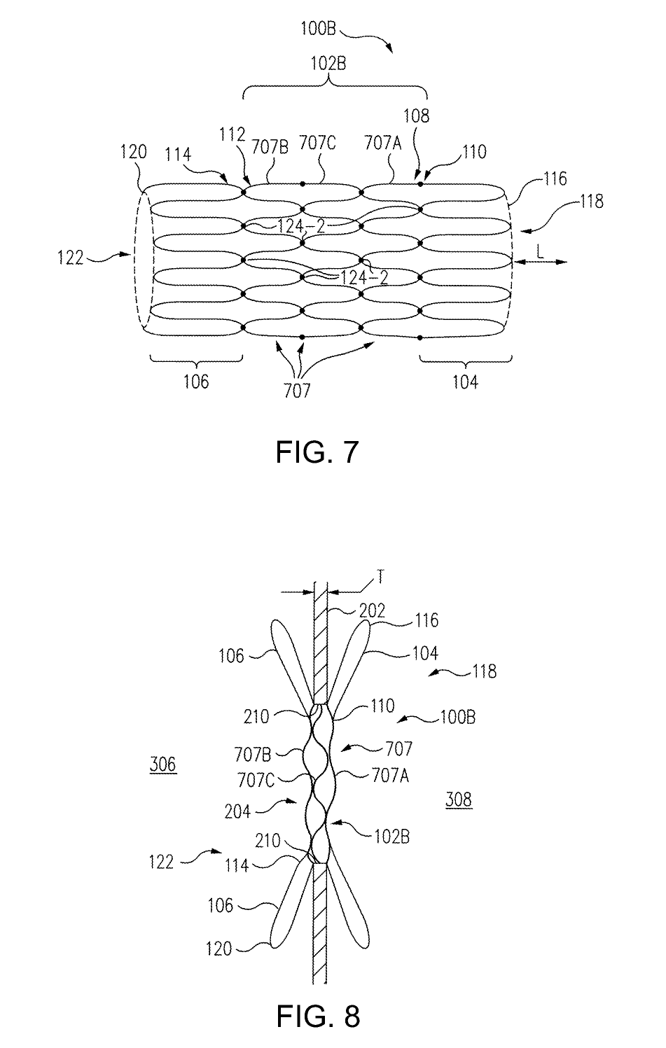

Although waist section 102 is illustrated as a single serpentine ring in FIG. 1, in another embodiment, a waist section is simply defined as the region of connection between first anchor section 104 and second anchor section 106 as discussed further below in reference to FIGS. 4, 5 and 6. It yet another embodiment, a waist section includes a plurality of interconnected serpentine rings as discussed further below in reference to FIGS. 7 and 8.

Further, although various expandable elements are described as serpentine rings, the expandable elements can be formed in other expandable patterns in other embodiments such as in a zigzag or diamond shaped pattern.

FIG. 3 is a partial cross-sectional view (similar to view in FIG. 2B) of another embodiment of a membrane eyelet 100-1 deployed within an opening 204 created in a tissue membrane 202 according to the present invention. In accordance with this embodiment, waist section 102 is a serpentine ring in an unexpanded configuration, but the waist section 102 can be fully expanded into an annular ring. Except for the deployed configuration of the waist section, the membrane eyelet depicted in FIG. 3 is very similar to the membrane eyelet depicted in FIGS. 1 through 2B. The membrane eyelet 100-1 includes a waist section 102 that is connected to a second anchor section 106 and a first anchor section 104.

In the embodiment depicted in FIG. 3, the annular ring created by the expansion of waist section 102 contacts the interior edge 210 of the tissue membrane 202 around the circumference of the opening that was created in the membrane. The annular ring allows for more contact with the edge of the opening than the contact with the struts 111 of the embodiment depicted in FIG. 2B.

FIG. 4 is a side view of a membrane eyelet 100A, prior to deployment, in one embodiment according to the present invention. In FIG. 4, only the near side cylindrical surface of membrane eyelet 100A is illustrated for clarity of illustration, however, it is to be understood that parts of the far side cylindrical surface of membrane eyelet 100A would also be visible.

As shown in FIG. 4, membrane eyelet 100A includes first anchor section 104 and second anchor section 106 as discussed above in reference to FIG. 1. However, in accordance with this embodiment, anchor sections 104, 106 are directly connected to one another by bridges 124-1, which define a waist section 102A. Bridges 124-1 extend between peaks 126 of the serpentine patterns of anchor sections 104, 106.

To illustrate, a first bridge 124A-1 of the plurality of bridges 124-1 extends between first peak 126A of the serpentine pattern of first anchor section 104 and a first peak 126B of the plurality of peaks 126 of the serpentine pattern of second anchor section 106.

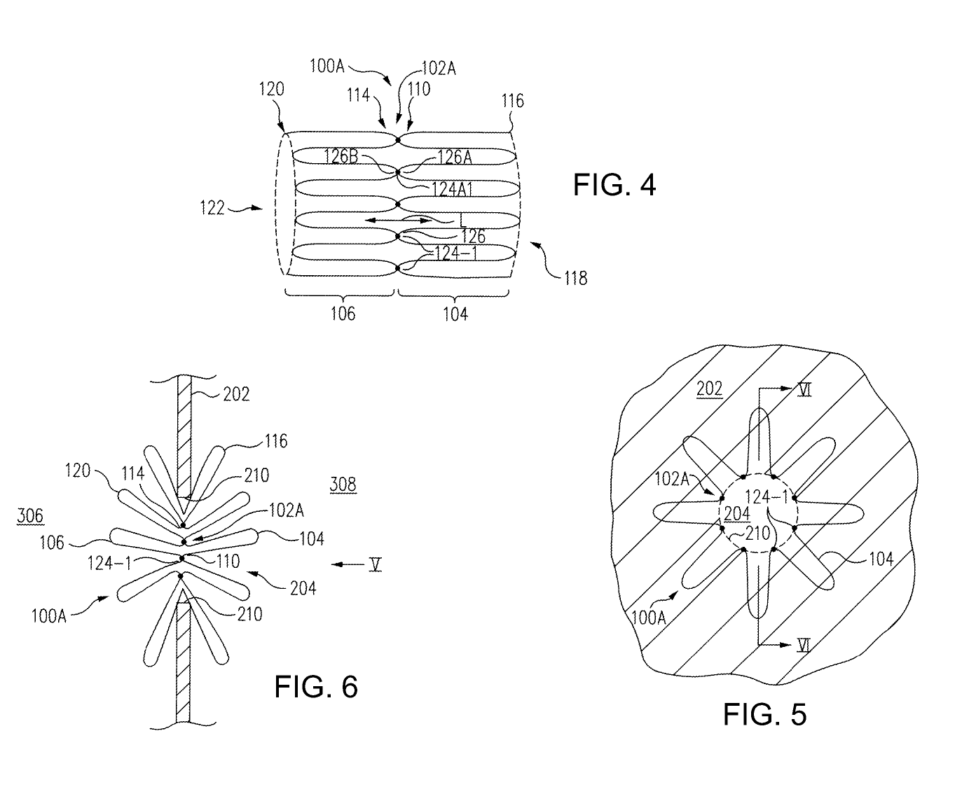

FIG. 5 is a front view of membrane eyelet 100A taken from the direction V of FIG. 6, after deployment within tissue membrane 202. FIG. 6 is a cross-sectional view taken at VI-VI of FIG. 5 of membrane eyelet 100A deployed within tissue membrane 202.

Referring now to FIGS. 5 and 6 together, bridges 124-1 directly contact the interior edge 210 at the edge of an opening 204 in the tissue membrane 202. More generally, the bridges create a waist section 102A which directly contacts interior edge 210 of the opening in the tissue membrane 202.

Bridges 124-1 prevent the surfaces of the interior edge 210 from contracting and thus prevents opening 204 from closing. Stated another way, bridges 124-1 keeps opening 204 open thus preventing constriction of the pathway through which fluid or air can pass from first region 306 to second region 308 or vice versa.

FIG. 7 is a side view of a membrane eyelet 100B, prior to deployment, in one embodiment according to the present invention. In FIG. 7, only the near side cylindrical surface of membrane eyelet 100B is illustrated for clarity of illustration, however, it is to be understood that parts of the far side cylindrical surface of membrane eyelet 100B would also be visible.

As shown in FIG. 7, membrane eyelet 100B includes first anchor section 104 and second anchor section 106 as discussed above. However, in accordance with this embodiment, anchor sections 104, 106 are directly connected by bridges 124-2 to a waist section 102B, which includes a plurality, e.g., three, of serpentine rings 707.

More particularly, first anchor section 104 is directly connected by bridges 124-2 to a first serpentine ring 707A of the plurality of serpentine rings 707. Second anchor section 106 is directly connected by bridges 124-2 to a second serpentine ring 707B of the plurality of serpentine rings 707. Serpentine rings 707A, 707B are directly connected by bridges 124-2 to a third serpentine ring 707C of the plurality of serpentine rings 707.

Although waist section 102B is illustrated and discussed above as including three serpentine rings 707A, 707B, and 707C, those of skill in the art will understand in light of this disclosure that a waist section can be formed having more or less than three interconnected serpentine rings.

FIG. 8 is a partial cross-sectional view of membrane eyelet 100B of FIG. 7, after deployment within tissue membrane 202. Referring now to FIG. 8, serpentine rings 707 directly contact interior edge 210 of tissue membrane 202. More generally, waist section 102B directly contacts interior edge 210 of tissue membrane 202.

Serpentine rings 707 prevent interior edge 210 from contracting and thus prevent opening 204 from closing. Stated another way, serpentine rings 707 keep opening 204 open thus preventing constriction of the pathway through which fluid or air can pass from first region 306 to second region 308 or vice versa.

Illustratively, by forming waist section 102B with a plurality of serpentine rings 707, waist section 102B is well suited to support interior edge 210 in the case when the thickness T of tissue membrane 202 is relatively large.

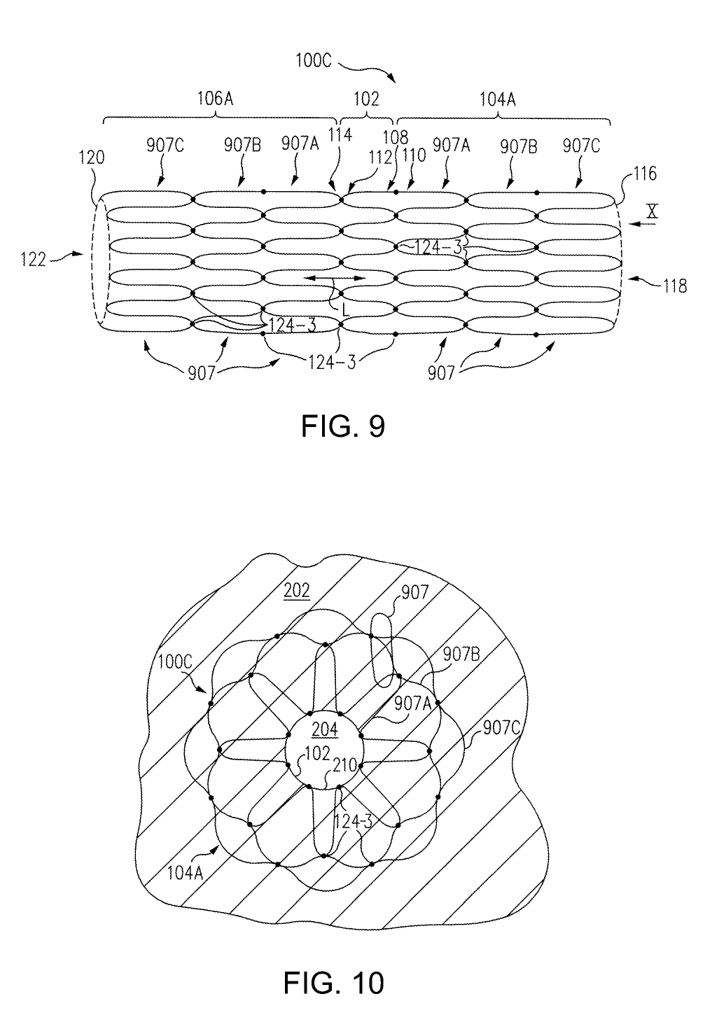

Although first anchor section 104 is illustrated as a single serpentine ring in FIG. 1, in another embodiment, first anchor section 104 includes a plurality of serpentine rings as discussed further below in reference to FIGS. 9 and 10.

FIG. 9 is a side view of a membrane eyelet 100C, prior to deployment, in one embodiment according to the present invention. In FIG. 9, only the near side cylindrical surface of membrane eyelet 100C is illustrated for clarity of illustration, however, it is to be understood that parts of the far side cylindrical surface of membrane eyelet 100C would also be visible.

As shown in FIG. 9, membrane eyelet 100C includes waist section 102 as discussed above in reference to FIG. 1. Waist section 102 is directly connected by bridges 124-3 to a first anchor section 104A and a second anchor section 106A. However, in accordance with this embodiment, anchor sections 104A, 106B each include a plurality, e.g., three, of serpentine rings 907.

More particularly, waist section 102 is directly connected by bridges 124-3 to a first serpentine ring 907A of the plurality of serpentine rings 907 of first anchor section 104A. First serpentine ring 907A is directly connected by bridges 124-3 to a second serpentine ring 907B of the plurality of serpentine rings 907 of first anchor section 104A.

Similarly, second serpentine ring 907B is directly connected by bridges 124-3 to a third serpentine ring 907C of the plurality of serpentine rings 907 of first anchor section 104A. Third serpentine ring 907C defines right edge 116 of first anchor section 104A and forms proximal end 118 of membrane eyelet 100C.

Further, waist section 102 is directly connected by bridges 124-3 to a first serpentine ring 907A of the plurality of serpentine rings 907 of second anchor section 106A. First serpentine ring 907A is directly connected by bridges 124-3 to a second serpentine ring 907B of the plurality of serpentine rings 907 of second anchor section 106A.

Similarly, second serpentine ring 907B is directly connected by bridges 124-3 to a third serpentine ring 907C of the plurality of serpentine rings 907 of second anchor section 106A. Third serpentine ring 907C defines left edge 120 of second anchor section 106A and forms distal end 122 of membrane eyelet 100C.

Although anchor sections 104A, 106A are illustrated and discussed above as each including three serpentine rings 907A, 907B, and 907C, those of skill in the art will understand in light of this disclosure that an anchor section can be formed having more, e.g., up to 50, or less than three interconnected serpentine rings.

FIG. 10 is a front view of membrane eyelet 100C viewed from the line X of FIG. 9, after deployment within tissue membrane 202. Referring now to FIG. 10, serpentine rings 907 of first anchor section 104A become progressively larger, i.e., have a larger average radial diameter, from first serpentine ring 907A to third serpentine ring 907C. Due to this progressive increase in size, once deployed, first anchor section 104A is sometimes said to be flower shaped. Illustratively, by using serpentine rings 907 having different properties, e.g., by forming serpentine ring 907C to be relatively thin and easily deformable compared to serpentine ring 907A, selective (more or less) flaring of first anchor section 104A is obtained. Second anchor section 106A is essentially identical in shape and function to first anchor section 104A and so is not illustrated or discussed for simplicity.

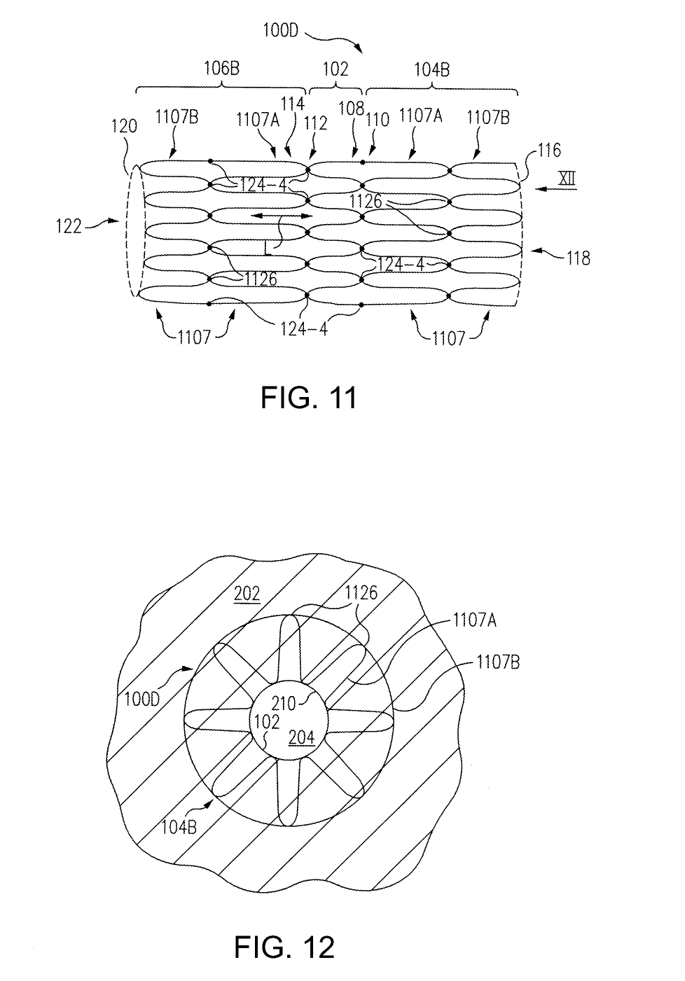

FIG. 11 is a side view of a membrane eyelet 100D, prior to deployment, in one embodiment according to the present invention. In FIG. 11, only the near side cylindrical surface of membrane eyelet 100D is illustrated for clarity of illustration, however, it is to be understood that parts of the far side cylindrical surface of membrane eyelet 100D would also be visible.

As shown in FIG. 11, membrane eyelet 100D includes waist section 102 as discussed above in reference to FIG. 1. Waist section 102 is directly connected by bridges 124-4 to a first anchor section 104B and a second anchor section 106B. However, in accordance with this embodiment, anchor sections 104B, 106B include a plurality, e.g., two, serpentine rings 1107.

More particularly, waist section 102 is directly connected by bridges 124-4 to a first serpentine ring 1107A of the plurality of serpentine rings 1107 of first anchor section 104B. First serpentine ring 1107A is directly connected by bridges 124-4 to a second serpentine ring 1107B of the plurality of serpentine rings 1107 of first anchor section 104B. Second serpentine ring 1107B defines right edge 116 of first anchor section 104B and forms proximal end 118 of membrane eyelet 100D.

Further, waist section 102 is directly connected by bridges 124-4 to a first serpentine ring 1107A of the plurality of serpentine rings 1107 of second anchor section 106B. First serpentine ring 1107A is directly connected by bridges 124-4 to a second serpentine ring 1107B of the plurality of serpentine rings 1107 of second anchor section 104B. Second serpentine ring 1107B defines left edge 120 of second anchor section 106B and forms distal end 122 of membrane eyelet 100D.

FIG. 12 is a front view of membrane eyelet 100D viewed from the line XII of FIG. 11, after deployment within tissue membrane 202. Referring to FIGS. 11 and 12 together, second serpentine ring 1107B of first anchor section 104B is sometimes called a stabilizing ring 1107B. More particularly, stabilizing ring 1107B becomes circularized, i.e., fully expanded into an annular ring, upon deployment of membrane eyelet 100D.

Stabilizing ring 1107B connects peaks 1126 of first serpentine ring 1107A thus providing stability and strength to first serpentine ring 1107A. Further, by enclosing peaks 1126 of first serpentine ring 1107A, stabilizing ring 1107B minimizes the possibility of the device used to deploy membrane eyelet 100D from catching on peaks 1126 of first serpentine ring 1107A and the associated unintentional detachment of membrane eyelet 100D from tissue membrane 202.

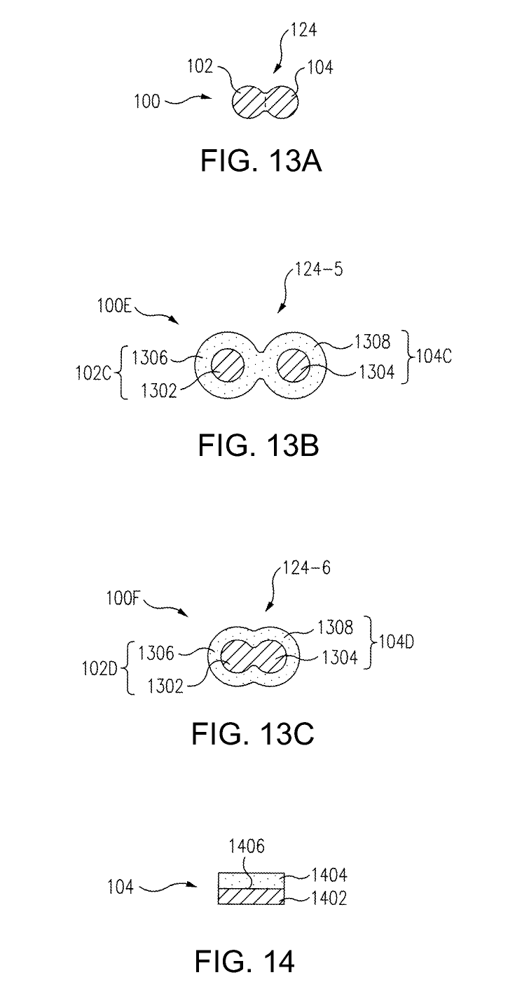

FIG. 13A is a cross-sectional view of bridge 124 of membrane eyelet 100 of FIG. 1 taken at XIII-XIII. In accordance with this embodiment, waist section 102 and first anchor section 104 are formed of the same material, e.g., a metallic, and this material is coupled, e.g., welded, fused, or otherwise joined, to form bridge 124.

FIG. 13B is a cross-sectional view of a bridge 124-5 of a membrane eyelet 100E similar to membrane eyelet 100 of FIG. 1. Waist section 102C and a first anchor section 104C are formed of a polymer coated metallic, e.g., a nylon coated steel.

More particularly, waist section 102C and first anchor section 104C include first and second metallic cores 1302, 1304 and first and second polymers 1306, 1308 enclosing and covering metallic cores 1302, 1304, respectively. Polymer 1306 of waist section 102C and polymer 1308 of first anchor section 104C are coupled, e.g., welded, fused, or otherwise joined, to form bridge 124-5. However, metallic cores 1302 and 1304 are not directly connected, but spaced apart.

FIG. 13C is a cross-sectional view of bridge 124-6 of a membrane eyelet 100F similar to membrane eyelet 100 of FIG. 1. A waist section 102D and a first anchor section 104D are formed of a polymer coated metallic, e.g., a nylon coated steel.

More particularly, waist section 102D and first anchor section 104D include metallic cores 1302, 1304 and polymers 1306, 1308 enclosing and covering metallic cores 1302, 1304, respectively. Polymer 1306 of waist section 102D and polymer 1308 of first anchor section 104D are coupled, e.g., welded, fused, or otherwise joined. Further, metallic core 1302 of waist section 102D and metallic core 1304 of first anchor section 104D are also coupled, e.g., welded, fused, or otherwise joined. Thus, bridge 124-6 is formed by the collective joining of polymer 1306, metallic core 1302 of waist section 102D to polymer 1308, metallic core 1304 of right anchor 104D, respectively.

Although a single bridge 124 is illustrated and discussed in FIG. 13A, in light of this disclosure, those of skill in the art will understand that the other bridges 124 of membrane eyelet 100 of FIG. 1 are formed similarly.

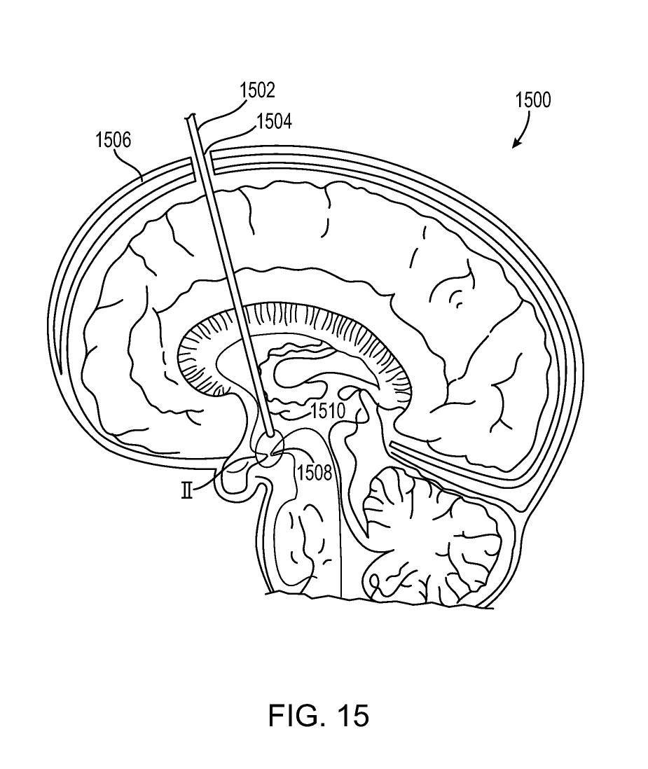

FIG. 14 is a cross-sectional view of membrane eyelet 100 of FIG. 1 taken at XIV-XIV. Membrane eyelet 100 is formed from a polymer-metallic laminate. Accordingly, membrane eyelet 100 is sometimes called a laminate structure.

More particular, membrane eyelet 100 includes a metallic core 1402 and a polymer 1404 on and coating a surface 1406 of metallic core 1402. Surface 1406 is either the outer cylindrical surface or the inner cylindrical surface of membrane eyelet 100.

A method according to the invention includes inserting a membrane eyelet into an opening of a membrane such that a waist section of the membrane eyelet is located in the opening and radially expanding the membrane eyelet such that the membrane is sandwiched between a first anchor section and a second anchor section of the membrane eyelet, where the step of radially expanding includes flaring the first anchor section and the second anchor section from the waist section, where the membrane can be the floor of the third ventricle.

Another method includes placing a membrane eyelet into an opening in the floor of the third ventricle. The membrane eyelet is deployed into the opening. The stent prevents the opening from closing. The membrane eyelet includes expanded ends that prevent the membrane eyelet from becoming disengaged from the floor.

FIG. 15 is a cross-section view of a human cranium 100 during an endoscopic third ventriculostomy (ETV) that would precede the implantation of a membrane eyelet according to the current invention. Initially, a burr hole 1504 is formed in the skull 1506. The probe 1502 is passed through burr hole 1504, through the cerebral cortex and through the underlying white matter to a location adjacent the floor 1508 of the third ventricle 1510 as illustrated in FIG. 15. The probe 1502 is then advanced through the floor 1508 of third ventricle 1510 to fenestrate floor 1508 and the underlying membrane of Lillequist. (The drawings show a rounded end, but other end configurations suitable for piercing may be used.) the procedure further includes measuring a flow of cerebrospinal fluid (CSF) with a flow sensor to check for proper fenestration. To complete the procedure, a membrane eyelet is deployed into the created opening.

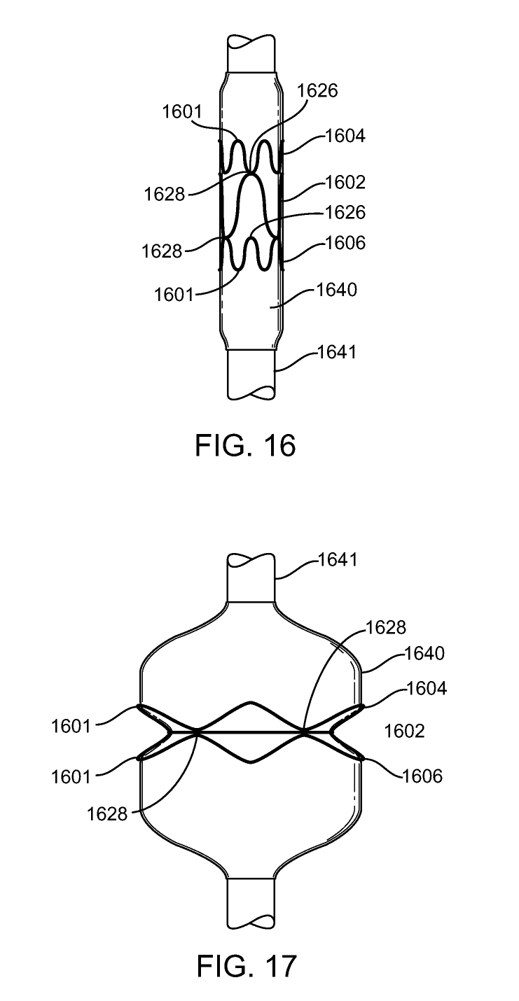

Referring to FIGS. 16 and 17, there can be illustrations of another embodiment of a membrane eyelet according to the current invention. FIG. 16 shows the membrane eyelet in a compressed delivery configuration on an expandable balloon. The membrane eyelet has a waist section 1602 that is coupled to a first anchor section 1604 and a second anchor section 1606. The anchor sections 1604, 1606 and the waist section 1602 are formed from generally serpentine rings. Waist section 1602 is between and directly coupled to first anchor section 1604 and second anchor section 1606.

Similar to the embodiments described in reference to FIGS. 1 through 3, waist section 1602 includes a first edge 108 coupled to a waist section edge of first anchor section 104 and a second, edge coupled to a waist section edge of second anchor section 106. The first and second edges of the waist section are defined by the ends of the serpentine ring, and a plurality of struts extend from the first edge to the second edge of the waist section. The first and second anchor sections each include an outer edge, having a plurality of outer peaks 1601.

Prior to deployment, as shown in FIG. 16, membrane eyelet is cylindrical in shape having a longitudinal axis. In the depicted embodiment, the waist section and the anchor sections all are serpentine rings wherein the serpentine pattern of the rings extends around a cylindrical surface. The first and second anchors are essentially identical and they are aligned such that the inner peaks 1626 of the first anchor section are directly opposite the inner peaks of the second anchor section and separated by the waist section.

In the embodiment depicted in FIGS. 16 and 17, the height of the struts created by the serpentine pattern of waist section are greater than the height of the struts created by the serpentine patterns of anchor sections. Additionally the waist has a pattern such that there are half as many peaks 1628 in the waist section as there are in the anchor sections. Thus, there is a bridge between a peak 1628 of the waist sections and every other inner peak 1626 for each of the anchor sections. Peaks 1626 and 1628 are sometimes called minima/maxima of the serpentine patterns of anchor sections 1604, 1606 and waist section 1602, respectively. Bridges 1624 can be formed at each adjacent peak 1626 and 1628, or only at some (fewer than all) of peaks 1626 and 1628.

Referring to FIG. 16, the membrane eyelet is in a delivery configuration wherein it is crimped to the surface of an expandable balloon 1640 that is disposed on an elongated delivery device 1641. Referring to FIG. 17, the membrane eyelet is then delivered to an opening in a tissue membrane (not shown for clarity) and the balloon 1640 is expanded to radially expand and deploy the membrane eyelet. Upon inflation of the balloon, the membrane eyelet is radially expanded and the shape of the balloon causes the anchor sections 1604 and 1606 to flare outward and engage the tissue membrane thus anchoring membrane eyelet to tissue membrane. The waist section 1602 is expanded into an annular ring configuration to engage the interior edges of the opening formed in the tissue membrane.

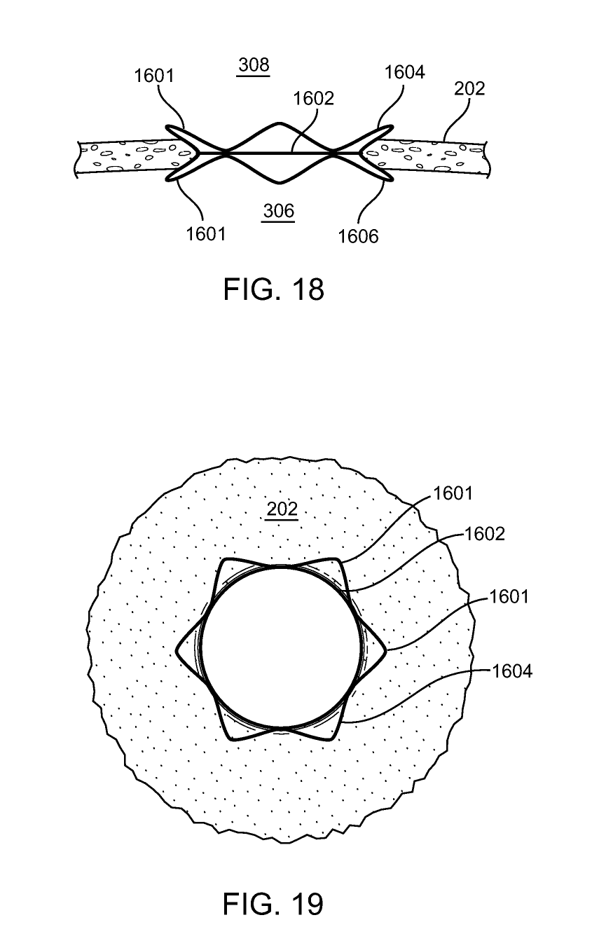

FIGS. 18 and 19 illustrate the membrane eyelet of FIG. 16 after it has been implanted in an opening that has been created in a tissue membrane 202 so that fluid or air can pass between a first region 306 and a second region 308. In the depicted embodiment, the tissue membrane 202 is secured between the outer peaks 1601 of the first and second anchor sections 1604, 1606. The annular ring formed by the waist section 1602 engages the interior edge of the opening in the tissue membrane to prevent the opening from closing. Fluid or air is then able to freely pass through the interior of the membrane eyelet.

In another embodiment, a membrane eyelet is self-expanding. In accordance with this embodiment, the membrane eyelet is constrained within a sheath (not shown). Retraction of the sheath exposes membrane eyelet, which self-expands. Use of a sheath to deploy a self-expanding device is well known to those of skill in the art and so is not discussed further.

Second anchor section 106B is essentially identical in shape and function to first anchor section 104B and so is not illustrated or discussed further for simplicity.

Embodiments of membrane eyelets of the current invention can be made from a single piece of material or they can be made from a plurality of separate pieces connected together. For example, in one embodiment of the current invention a membrane eyelet is formed by laser cutting a tubular piece of material. However, in an alternative embodiment, a waist section, a first anchor section, and a second anchor section are separate pieces, which are connected together, e.g., by welding.

The membrane eyelets of the current invention can be formed from any biocompatible material having suitable shape memory properties. Various embodiments of the membrane eyelets of the current invention can be from materials selected from a group that includes but is not limited to: 1) stainless-steel; 2) chromium alloy; 3) a shape memory alloy such as nickel titanium that has been heat-set, or tempered, in such a manner to provide a membrane eyelet with an inherent self-expanding characteristic; and/or 4) polymer; and/or 5) a combination thereof. One embodiment of a membrane eyelet according to the current invention includes a waist section and anchor sections that are formed from the same material. Another embodiment of a membrane eyelet includes anchor sections that are formed from different material that the waist section.

This application is related to Stiger et al., U.S. patent application Ser. No. 10/423,144 entitled “FLOW SENSOR DEVICE FOR ENDOSCOPIC THIRD VENTRICULOSTOMY”, the entirety of which is herein incorporated by reference thereto.

This disclosure provides exemplary embodiments of the present invention. The scope of the present invention is not limited by these exemplary embodiments. Numerous variations, whether explicitly provided for by the specification or implied by the specification or not, such as variations in structure, dimension, type of material and manufacturing process may be implemented by one of skill in the art in view of this disclosure.

What is claimed is:

1. A membrane eyelet for deployment in an opening in a tissue membrane comprising:
an expandable waist section;
an expandable first anchor section and an expandable second anchor section;
the first anchor section, and the second anchor section each having a waist end that is coupled to the waist section and a free end with a long axis extending therebetween;
the membrane eyelet having a delivery configuration in which the waist section, the first anchor section and the second anchor section are each a generally serpentine ring; and
the membrane eyelet having a deployment configuration in which the waist section is an annular ring and the free ends of the anchor sections extend at an angle from the long axis of the anchor sections.

2. The membrane eyelet of claim 1 wherein when the membrane eyelet is in the delivery configuration, it has a cylindrical shape such that each of the anchor sections have a radial delivery diameter and the long axis of each of the anchor sections is parallel to the long axis of the other anchor section.

3. The membrane eyelet of claim 2, wherein the waist section, the first anchor section and the second anchor section have a same radial delivery diameter.

4. The membrane eyelet of claim 1 wherein when the membrane eyelet is in the deployment configuration, each of the free ends have a radial deployment diameter that is greater than the radial delivery diameter, the free end of each of the anchor sections extends at an angle from the long axis of the membrane eyelet, the waist end of each of the anchors has a radial deployment diameter that is smaller than the radial deployment diameter of the free ends of the anchor sections, and the waist section has a smaller radial diameter than the radial deployment diameter that is smaller than the radial deployment diameter of the free ends of the anchor sections.

5. The membrane eyelet of claim 4 wherein the radial deployment diameter of the waist ends of each anchor section is the same, and the waist section has a radial deployment diameter that is equal to the radial deployment diameter of the waist ends of the anchor sections.

6. The membrane eyelet of claim 1 wherein the waist section is coupled to the first anchor section and the second anchor section with a plurality of bridges.

7. The membrane eyelet of claim 1 wherein when the membrane eyelet is in a deployment configuration, the first anchor section has an increasing radial diameter between the first edge of the first anchor section and the second edge of the first anchor section.

8. The membrane eyelet of claim 1 wherein an angle between the first anchor section and a longitudinal axis of the membrane eyelet is less than 90°.

9. The membrane eyelet of claim 8 wherein the first anchor section defines a conical surface.

10. A structure comprising:
a membrane eyelet configured for insertion in an opening in a tissue membrane in the body of a patient, the membrane eyelet further comprising;
an expandable waist section for engaging an interior edge of an opening in a tissue membrane;
a first anchor section coupled to the waist section;
a second anchor section coupled the waist;
the first anchor section, and the second anchor section each having a long axis;
the first anchor section, and the second anchor section each having a waist end that is coupled to the waist section and a free end;
the membrane eyelet having a delivery configuration in which each of the anchor sections have a radial delivery diameter and the long axis of each of the anchor sections is parallel to a long axis of the waist section; and
the membrane eyelet having a deployment configuration in which the free end of each of the anchor sections is flared radially outward such that the free ends have a radial deployment diameter that is greater than the radial delivery diameter, the free end of each of the anchor sections extends at an angle from the long axis of the waist section and the waist section has a smaller radial diameter than the radial deployment diameter of the free ends of the anchor section, and the waist section being expanded into an annular ring such that when the structure is inserted in an opening in a tissue membrane in the body of a patient the anchor sections are outside of the opening and each is on a different side of the tissue membrane, the tissue membrane is secured between the anchor sections and the annular waist is positioned to engage an inner edge of the opening in the tissue membrane.

11. The membrane eyelet of claim 10, wherein the waist section, the first anchor section and the second anchor section have a same radial delivery diameter.

12. The membrane eyelet of claim 14 wherein the waist section, the first anchor section, and the second anchor section are serpentine rings when they are in the delivery configuration.

13. The membrane eyelet of claim 12 wherein when the waist section is in a delivery configuration, the waist section comprises a serpentine ring having a first end and a second end with a plurality of struts extending therebetween.

14. The membrane eyelet of claim 12 wherein each of the anchor sections comprises a plurality of serpentine rings.

15. The membrane eyelet of claim 10 wherein a radial deployment diameter of the waist ends of each anchor section is the same, and the waist section has a radial deployment diameter that is equal to the radial deployment diameter of the waist ends of the anchor sections.

16. The membrane eyelet of claim 10 wherein the waist section is coupled to the first anchor section and the second anchor section with a plurality of bridges.

17. The membrane eyelet of claim 10 wherein when the membrane eyelet is in a deployment configuration, the first anchor section has an increasing radial diameter between the first edge of the first anchor section and the second edge of the first anchor section.

18. The membrane eyelet of claim 10 wherein an angle between the first anchor section and a longitudinal axis of the membrane eyelet is less than 90°.

19. The membrane eyelet of claim 18 wherein the first anchor section defines a conical surface.

20. A membrane eyelet for eyelet for deployment in an opening in a tissue membrane comprising:
an expandable waist section having a delivery configuration in which the waist section is a serpentine ring and a deployment configuration in which the waist section is an annular ring;
an expandable first anchor section and an expandable second anchor section, each anchor section having a free end, a waist end that is coupled to the waist section by a plurality of bridges and a long axis between the free end and the waist end; and
each of the anchor sections having a deployment configuration in which the free end of the anchor section has a radial diameter that is greater than the radial diameter of the waist end of the anchor section.
